(19) United States
(12) Patent Application Publication
(10) Pub. No.: US 2004/0118400 A1
(43) Pub. Date: Jun. 24, 2004

(54) Humidifier to adjust of air into nasal cavity

(76) Inventor: Chojen Chou, Shindian City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/327,010

(22) Filed: Dec. 24, 2002

(51) Int. Cl. A61M 15/00

(52) U.S. Cl. 128/204.12

(57) ABSTRACT

A humidifier uses a moist member to adjust humidity of the air breathed in. In general, the people work in low humidity atmosphere would breathe un-smoothly and get the respiratory system illness more easily. So the humidifier greatly improves the situation described above. The moist member is a bibulous material. Put the moist member between nose and top lip to adjust the humidity of the air breathed in by vaporizing the liquid of the moist member.

### BACKGROUND OF THE INVENTION

1. Field of the Invention

The present invention relates to a humidifier to adjust of air into nasal cavity for people breathe in, and more particularly, a humidifier has a moist member for adsorbing liquid to control humidity into nasal cavity.

2. Description of the Related Art

People work in low humidity atmosphere or dry area, like airplane pilot in airplanes cabin, is very uncomfortable, especially on duty over 10 to 20 hours. Humidity of atmosphere is almost 0 in airplanes cabin, so people feel uncomfortable and need to keep moisture. In general, people use wet towel to cover nose and mouth or place nearby for increasing humidity, so as to feel comfortable and sleep well, or else the people has found some blood clot in nasal cavity. Further, if people, who go to different countries, stand different climate in huge changes is difficulty, and get the respiratory system illness, like catching a cold or nasal disease, more easily.

According to medicine report, people live in the highest humidity area or working in the lowest humidity area, to catch the respiratory system disease is easily.

The present invention provides a humidifier to improve humidity of air, especially when humidity of atmosphere is 0, people are breathing un-smoothly. The humidifier of present invention is base on many years of experience of research, development, and sales in relative field bring about the humidifier to solve problem as above described.

### SUMMARY OF THE INVENTION

It is therefore an object of the present invention is to provide a humidifier which uses a moist member to adjust humidity of air into nasal cavity. The liquid of moist member is taken away by air so as to adjust humidity of air.

It is an object of the present invention is to provide a humidifier which uses a net with different sizes of mesh to control speed of the liquid vaporized by air from moist member so as to control humidity of air into nasal cavity.

It is another object of the present invention to provide a humidifier has a T shape moist member for absorbing medicine to cure nasal disease directly.

It is a further object of the present invention is to provide a humidifier which has a reservoir connecting to a moist member for continuing to absorb liquid for prolonging time of validity of moist member.

The present invention relates to a humidifier for adjusting humidity and temperature. In general, people who work in low humidity or dry area like airplane pilot in airplanes cabin always feel uncomfortable, and infect disease easily. A humidifier of present invention can keep humidity and or prevent to infect disease and feel comfortable. Further, the humidifier of present invention can help people breathe in fresh air and prevent bacterial infection and remedy chronic nasal disease. The humidifier is made of bibulous material. The air through the moist member vaporizes liquid from moist member to nasal cavity to adjust humidity of air for improving breath smoothly.

Further, the moist membrane as above described can add a net to control vaporizing liquid for adjusting the humidity of air into nose. The net also has different mesh sizes for efficacious controlling speed of vaporizing.

Furthermore, the moist membrane is formed a T shape. The T shape moist membrane also can be put inside nasal cavity to cure nasal disease. The reservoir as above described connects to the T shape moist membrane can prolong treatment period.

### BRIEF DESCRIPATION OF THE DRAWINGS

FIG. 1 shows top view of humidifier of present invention;

FIG. 2 shows a cross-section view of present invention;

FIG. 3 shows one of embodiment of present invention;

FIG. 4 shows a cross section in front view of embodiment of present invention;

FIG. 5 shows a cross section in side view of embodiment of present invention;

FIG. 6 shows another of embodiment of present invention;

FIG. 7 shows a side view of embodiment with reservoir of present invention; and

FIG. 8 shows a front view of embodiment with reservoir of present invention.

### DETAILED DESCRIPATION OF PREFERRED EMBODIMENTS

In order to understand the present invention, reference is made to the accompanying drawings. Understanding that these drawings are not to be considered limitations in the scope of the invention, the presently preferred embodiments and the presently understood best mode of the invention are described with additional detail through use of the accompanying drawings in which:

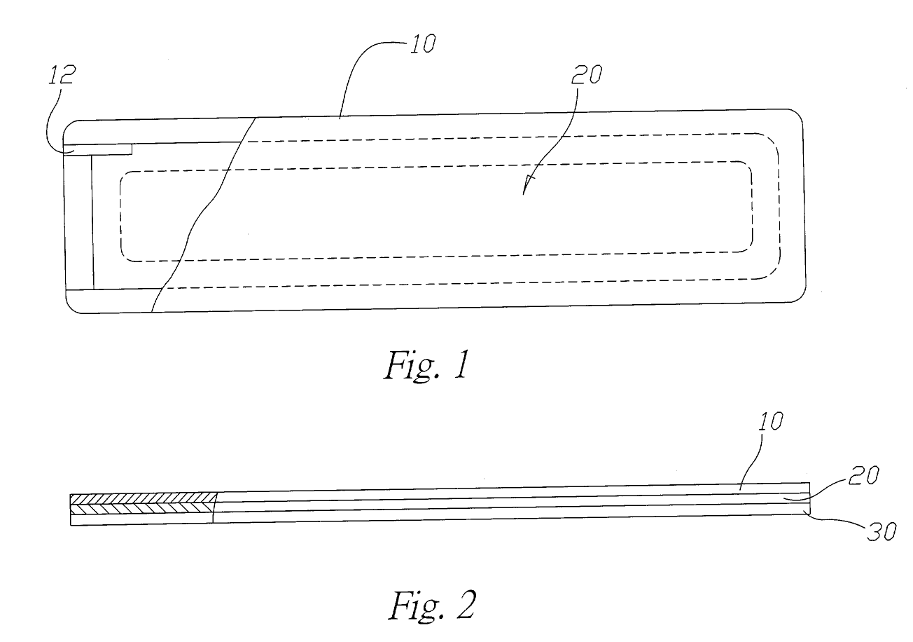

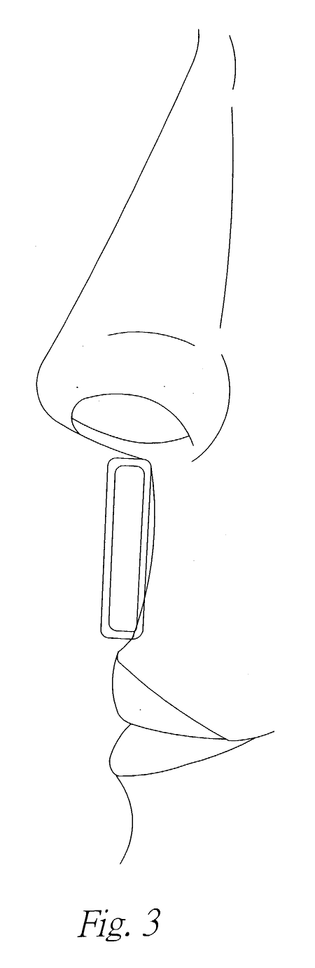

Referring to FIGS. 1 and 2 shows a top view and a cross-section view of present invention; the humidifier of present invention comprises a base 10 and a moist member 20. The base has an adhesive layer for sticking between nose and mouth. Referring to FIG. 3, the moist member 20 is placed under the base 10. The base 10 is made of waterproof materials like non-woven fabric or plastic. The moist member 20 is made of bibulous materials for absorbing liquid. When people breathe in air through the moist member 20, the air brings liquid from the surface of moist member 20 in nasal cavity. The liquid can adjust humidity of atmosphere for breathing comfortable. For patient with nasal disease, the moist member 20 also can be added medicine to cure disease.

Further, the moist member 20 of present invention also has an injecting open 12 and a net 30. The injecting open 12 is use of adding liquid into the moist member 20 any time. The net 30 has different size of meshes to control the liquid be vaporized from moist member 20. The air through the different size of meshes of net takes away the liquid on moist member 20 so as to adjust humidity of air precisely into people's nose, so the people breathe comfortable.

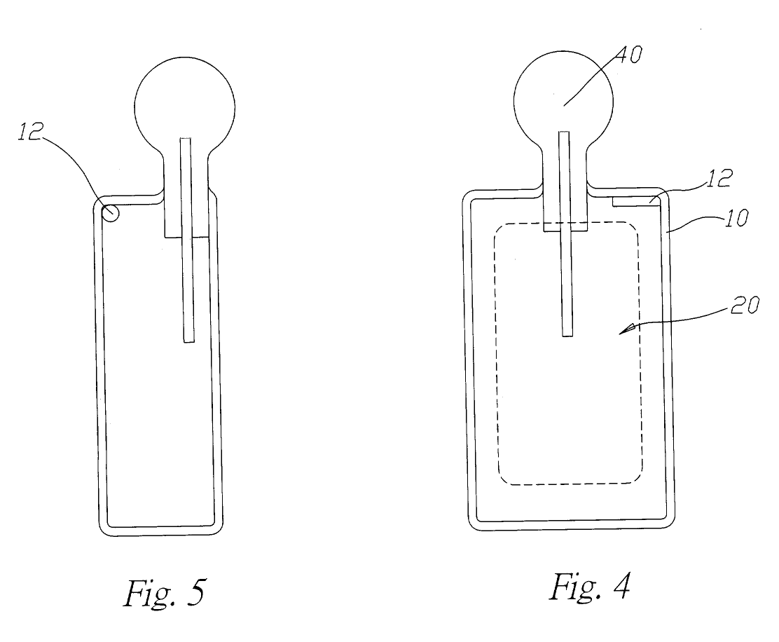

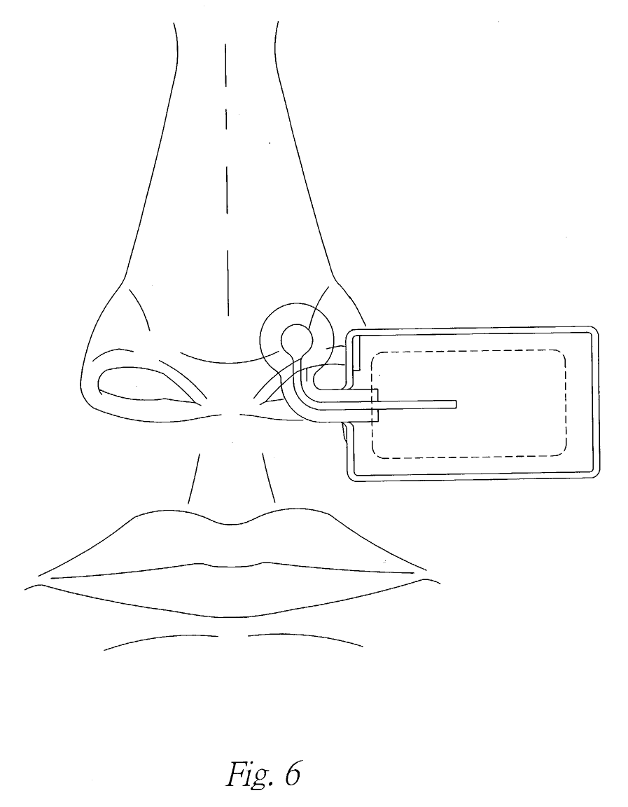

Referring to FIGS. 4 and 5 shows a cross section in front view and a cross section in side view of embodiment. The moist member is a T shape moist member 40, one of end of T shape can be put inside nasal cavity show in FIG. 6. The T shape moist member 40 absorbs medicine to cure nose disease.

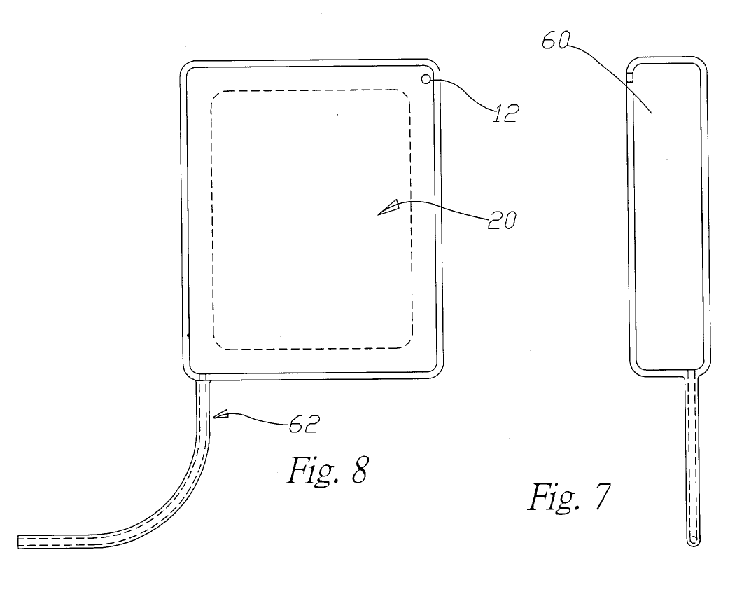

Referring to FIGS. 7 and 8 shows a side view and a front view of embodiment with reservoir of present invention. The moist member also has a reservoir 60 with a connector 62. The connector 62 connects to injecting open 12 of the moist member for supplying liquid continually in treatment time. The reservoir 60 supplies the liquid in accordance with principle of position energy.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A humidifier for adjusting humidity of air into nasal cavity comprising:
a base having an adhesive surface in lower side to stick on proper place between nose and mouth; and
a moist member connecting top of said base.

2. The humidifier according to claim 1, wherein said base is made of waterproof materials.

3. The humidifier according to claim 1, wherein said base is made of non-woven fabric.

4. The humidifier according to claim 1, wherein said moist member is made of bibulous materials.

5. The humidifier according to claim 1, wherein said moist member comprises a net above.

6. The humidifier according to claim 5, wherein the mesh of net controls the speed of vaporized by different size of mesh.

7. The humidifier according to claim 1, wherein said moist member has a reservoir with a connector connecting to.

8. The humidifier according to claim 7, wherein said reservoir places higher than said moist member.

9. The humidifier according to claim 7, wherein said reservoir comprises an adhesive layer.

10. A humidifier for adjusting humidity of air into nasal cavity comprising:
a moist member having an adhesive layer in lower side to stick on proper place between nose and mouth.

11. The humidifier according to claim 10, wherein said moist member is made of bibulous materials.

12. The humidifier according to claim 10, wherein said moist member has a net above.

13. The humidifier according to claim 12, wherein said moist member has a net with mesh for controlling speed of vaporized according to different size of mesh.

14. The humidifier according to claim 10, wherein said moist member has a reservoir with a connector connecting to.

15. The humidifier according to claim 14, wherein said reservoir places higher than said moist member.

16. The humidifier according to claim 14, wherein said reservoir comprises an adhesive layer.
